(19) United States
(12) Patent Application Publication
(10) Pub. No.: US 2024/0261114 A1
(43) Pub. Date: Aug. 8, 2024

(54) APPARATUS AND METHODS FOR TOTAL HIP REPLACEMENT BROACH AND STEM INSERTION

(71) Applicant: Value Forces Ltd., Haifa (IL)

(72) Inventors: Nir KARASIKOV, Haifa (IL); James DICK, Muntinlupa City (PH)

(73) Assignee: Value Forces Ltd., Haifa (IL)

(21) Appl. No.: 18/566,651

(22) PCT Filed: Jun. 15, 2022
(86) PCT No.: PCT/IL2022/050646

§ 371 Date: Dec. 3, 2023

Related U.S. Application Data
(60) Provisional application No. 63/210,589, filed on Jun. 15, 2021.

(51) Int. Cl. A61F 2/46; A61B 17/92

(52) U.S. Cl. CPC A61F 2/4607; A61B 17/92; A61B 2017/924; A61F 2002/4666; A61F 2002/469

(57) ABSTRACT

An apparatus and method for inserting a broach or stem into a bone, the broach and stem having an associated handle for impaction. The apparatus includes: a closed loop impactor configured to provide a series of impacts on the handle: at least one sensor configured to sense the series of impacts, including sensing the bone stress and to provide real-time feedback of a condition of the bone to the impactor's impact force, displacement and impact frequency; and a processor operably connected to the at least one sensor and configured to receive the real-time feedback therefrom, and operably connected to the impactor to control the rate and force of the impacts that the impactor applies to the broach.

### CROSS-REFERENCE TO RELATED APPLICATIONS

This application claims priority from U.S. Provisional Patent Application Ser. No. 63/210,589, filed on 15 Jun. 2021, the entire contents of which are incorporated herein by reference.

### TECHNOLOGICAL FIELD

The presently disclosed subject matter is in the field of orthopedics, in particular hip replacements.

### BACKGROUND

A Total Hip Replacement (THR) offers excellent results in patients suffering from end-stage osteoarthritis (OA). During total and partial hip replacement, a surgeon implants a femoral stem. There are two ways to fix the femoral stem to the femur bone, organic (cementless), which is by far the most common procedure, and cemented.

With cementless stem fixation, the surgeon inserts broaches of gradually increasing size into the femoral canal, until getting the correct size broach, which is replaced by an artificial femoral stem upon which a ball joint is fitted. The cementless stem fixation is based on a press-fit mechanism, the preparation of the medullary femoral canal is 1.5-2 mm smaller than the natural stem.

To insert the broaches, the broaches are contacted by a broach handle, which is repeatedly impacted with a surgical mallet. A series of broaches are used, starting from an initial small broach to successively larger broaches. If the broach is impacted with excessive force, the femur bone might fracture.

The impact strength is presently controlled subjectively, for example, by the sound emitted by a mallet/hammer blow and femur. There is no precise control of the impact intensity and no feedback regarding the bone condition other than the impact noise and the physician's experience and intuition.

A major issue in hip replacements, in particular with total hip arthroplasty (THA) using cementless femoral stems, is the occurrence of periprosthetic femoral fractures. Press-fit impaction has been the most popular technique for the fixation of cementless femoral stems, which may lead to the periprosthetic femoral fractures. The incidence of periprosthetic femoral fractures with cementless femoral stems during primary THA has been reported to be 3.5-5.4%. The incidence of periprosthetic femoral fractures during ‘revision’ THA is reported to be in the range of 13% to 21%. The incidence of ‘revision’ THA is reported to be 8% of all ‘primary’ THA procedures.

Related methods in the art include a jack hammer type device to replace the manual hammer, as illustrated in https://www.youtube.com/watch?v=08LMFzQuuaE; and audible sensing by the surgeon of the change in the noise, when the broach touches the hard bone cortical perimeter.

Publications relating to issues and experimentation regarding femoral stem insertion include:

Sakai, et al., “Hammering force during cementless total hip arthroplasty and risk microfracture”, Hip Int 2011; 21 (03) 330-335.

Krull, et al., “Maximizing the fixation strength of modular components by impaction without tissue damage”, Bone and Joint Research, vol. 7, No. 2, 196-204, 1 Feb. 2018.

Greenhill, et al. “Broach handle design changes for distribution in the femur during total hip arthroplasty”, The Journal of Arthroplasty 32 (2017) 2017-2022.

Tijou, et al., “Monitoring cementless femoral stem insertion by impact analyses: an in vitro study”, J Mech Behav Biomed Mater, 2018 December; 88:102-108, doi: 10.1016/j.jmbbm.2018.08.009, Epub 2018 Aug. 10.

Tijou, et al., “Study Monitoring cementless femoral stem insertion by impact”, Journal of mechanical behavior of biomedical materials, Elsevier, 2018, https://arxiv.org/abs/1905.08246.

Additional publications relevant to the technology include: U.S. Pat. No. 10,463,505 (Behzadi); U.S. Pat. No. 9,430,189 (Soles, et al.); US 2018/228614 (Lang, et al.); and WO 2021/174295 (Miles, et al.).

### GENERAL DESCRIPTION

The presently disclosed subject matter relates to a method and apparatus to help assure appropriate impacting on a broach handle to insert a broach (artificial bone stem) into the femoral bone so that the broach will progress and firmly position itself and yet not fracture the bone. A proper cementless stem insertion allows a stable stem-bone connection and good bone ingrowth, and avoids fractures around the implanted stem, which can also occur if the broach is too big or impacted too hard. On the other hand, if the broach is too loose, there will be a subsidence and the effective bone length will shorten, and the implant will be unstable.

The presently disclosed subject matter provides an apparatus including an impact sensor; an impactor; and a processor, with an impact analysis algorithm. The sensor may be any of (or combination of) a variety of sensors including accelerometer; ultrasonic; microphone; vibrometer; interferometer or position sensor; acoustic emission sensor; and acoustic transducer, which transmits and receives acoustic radiation. In some examples, the sensor is located between the broach or stem and the impacting handle. The impactor is configured to impact on the broach handle to mechanically insert the broach into the bone, in particular, in a controllable and quantifiable manner.

The processor is configured to receive output from the sensor(s), to analyze the output of the sensor(s), and to control the impacting of the impactor, typically using a continuous feedback from the sensor by analysis of shock waves (e.g. acoustic waves) produced during the impacting by the impactor on the broach handle. In the case of sensor 50 being constituted by a transducer, a chirp wave could be generated right after the impact and reflected waves are detected by the same, or another/auxiliary, transducer. The reflected waves include information about the bone stress (e.g. through bone acoustic impedance) and provide an input to the impact analysis algorithm. The analysis may include comprehensive deep learning algorithms to learn the fingerprint of a bone to assure optimal insertion through feedback from the bone (i.e. via the sensor) so when the broach approaches the correct insertion position, the surgeon will receive an indication to lower the impact intensity; or the processor's analysis algorithm will instruct the processor to signal the impactor to lower the impact intensity.

In some examples of the presently disclosed subject matter, the impactor is operably connected to the processor and controlled thereby. This control of the impactor by the processor, based on signals from the sensor(s), prevents use of excessive impact force. A subset of this feature is where the apparatus, via the impactor, provides impacting above a threshold impact force so that the broach will progress into the bone canal (i.e. initially above an elastic spring-back force and deflection of the soft tissue, and then on the cortex tissue), but within a range of impact force that ensures that the bone will not fracture.

In particular, the presently disclosed subject matter provides an apparatus for inserting a broach or stem into a bone, the broach and stem having an associated handle for impaction, the apparatus including: a closed loop impactor configured to provide a series of impacts on the handle; at least one sensor configured to sense the series of impacts, including the force and vibration frequency of the impacts and to provide real-time feedback of a condition of the bone; and a processor operably connected to the at least one sensor and configured to receive the real-time feedback therefrom, and operably connected to the impactor to control the rate and force of the impacts that the impactor applies to the broach.

In some examples, the sensor is any one or combination of an accelerometer, a vibrometer, an interferometer or position sensor, a wide band microphone, an acoustic emission sensor, and a transducer.

In some examples, the sensor is configured to provide real-time feedback based on shock-induced vibrational data or acoustic data.

In some examples, the processor includes an artificial intelligence system for multi-parameter data analysis.

In some examples, the impactor is a piezoelectric impactor.

In some examples, the impactor is an electro-magnetic impactor.

In some examples, the impactor is configured to provide impacts at a frequency of between 10 and 100 impacts per second.

In some examples, the impactor includes a moving mass, a spring, a hitting mass and an excitation device.

In some examples, the excitation device is either a motor; a solenoid; or a voice coil.

In some examples, the at least one sensor is disposed between the broach handle and the broach.

In some examples, the at least one sensor is disposed on one or more sides of the broach handle and/or the broach.

In some examples, the at least one sensor is disposed on or adjacent a corresponding knee.

In some examples, the impactor is configured to impact the broach or bone at a rate of 1 Hz to 100 Hz.

The presently disclosed subject matter also provides a method of inserting a broach/stem into a bone with real-time feedback. The method includes (a) measuring shock waves produced from impacting on a broach (i.e., measuring the shock waves produced by the broach being inserted into the bone by an impactor impacting on a broach handle thereof) or by generating acoustic waves in the broach by a transducer at variable frequency; (b) analyzing the shock waves and/or acoustic waves via an algorithm operated by a processor to derive the bone stress; and (c) providing feedback to the impactor so as to control the force and or rate of impacting.

In particular, the presently disclosed subject matter provides a method of inserting a broach or stem into a bone using an impactor, the method including: impacting the broach or stem into the bone; using at least one sensor to sense signals (a) produced by the broach or stem during said impacting, or (b) signals generated in the bone as a result of the impacting; analyzing said signals via an algorithm operated by a processor; and providing real-time feedback to control the force and rate of impacting.

In some examples, the sensor is an acoustic emission sensor, configured to sense acoustic signals related to the generation of micro-cracks in the bone, and signal sensed by the acoustic emission sensor is filtered to differentiate between the impact signal and the signal from the tissue and bone.

In some examples, the impacting is performed so that subsequent impacts are within the transient time of a previous impact, thereby resulting in a dynamic coefficient of friction to lower friction between the broach or stem and the bone.

In some examples, the impacting includes using an electro-magnetic impactor and/or a piezo impactor.

In some examples, the impacting includes using an inertial impactor having a lower/impacting mass and an upper mass designed to move in opposite directions where only the lower mass operably impacts the broach or stem.

In some examples, the impacting is performed at a rate of 1 Hz to 100 Hz.

In other words, the apparatus and method are configured to monitor the broach and stem insertion, i.e. monitor the impact so as to reduce the chances of bone fracture; and determine an impact force and movement/displacement of the broach or stem, to assure that the displacement is large enough to exceed the elastic response of the bone's tissue and assure penetration (i.e. above a “spring-back” elastic bone response), to ensure the broach proceeds into the bone while not exceeding a stress determined by the processor, to mitigate the chance of fracture, while penetrating into the bone's cortex (harder tissue).

It is a particular feature of the presently disclosed subject matter that the desired impact on the broach or stem is determined using shock or acoustic waves as detected by the sensor(s), and shock wave analysis.

It is another particular feature of the presently disclosed subject matter to provide for impaction at a higher rate than manual impacting/hitting, allowing for impacting at a lower intensity to perform the same penetration. Preferably, a subsequent impact is within the transient period that occurs while the broach or stem is still vibrating due to the previous impact, or the shock waves generated from the impact are still not damped, so that the coefficient of friction (COF) between the bone and the implant is dynamic and not static, providing for a more efficient broach insertion. With a high impact rate, the force required is lower as the overall insertion of the broach or stem occurs in multiple small steps. Another beneficial result is that the broach/stem movement can be dynamic, whereby the coefficient of friction (COF) is lower.

### BRIEF DESCRIPTION OF DRAWINGS

The presently disclosed subject matter may be more clearly understood upon reading of the following detailed description of non-limiting examples thereof, with reference to the following drawings, in which:

FIG. 1 is a schematic perspective view of an apparatus for inserting a broach or stem in a bone, in accordance with an example of the presently disclosed subject matter.

FIG. 2 illustrates an exemplary sensor of the apparatus, exemplified by an accelerometer, located on the shin of a patient, in accordance with an example of the presently disclosed subject matter.

FIG. 3 is a schematic perspective view of a broach; a broach handle; a sensor; and a sensor holder, in accordance with an example of the presently disclosed subject matter.

FIGS. 4A and 4B are schematic perspective view of exemplary ultrasonic transducers/sensors of the apparatus, in accordance with an example of the presently disclosed subject matter.

FIG. 5 is a schematic front view of most of the apparatus, illustrating an exemplary impactor thereof, in accordance with an example of the presently disclosed subject matter.

FIG. 6 is a side view of another exemplary impactor of the apparatus, in accordance with an example of the presently disclosed subject matter.

FIG. 7 is a graph depicting the relation between voltage, stroke, and impact force of the impactor of FIG. 6.

FIG. 8 is a snap-shot of a simulation of an impact on a broach/stem, showing bending vibration waves in the broach/stem and the bone.

FIG. 9 is a snap shot of a simulation video showing bone reaction to the broach upon an impact, Von Mises stress is shown.

FIG. 10 is a graph of wave vibration measured in decibels as a function of frequency in a longitudinal and bending mode.

The following detailed description of examples of the presently disclosed subject matter refers to the accompanying drawings referred to above. Dimensions of components and features shown in the figures are chosen for convenience or clarity of presentation and are not necessarily shown to scale. Wherever possible, the same reference numbers will be used throughout the drawings and the following description to refer to the same and like parts.

### DETAILED DESCRIPTION

Illustrative examples of a broach/stem insertion/impacting apparatus according to the presently disclosed subject matter are described below. In the interest of simplicity, not all features/components of an actual implementation are necessarily described.

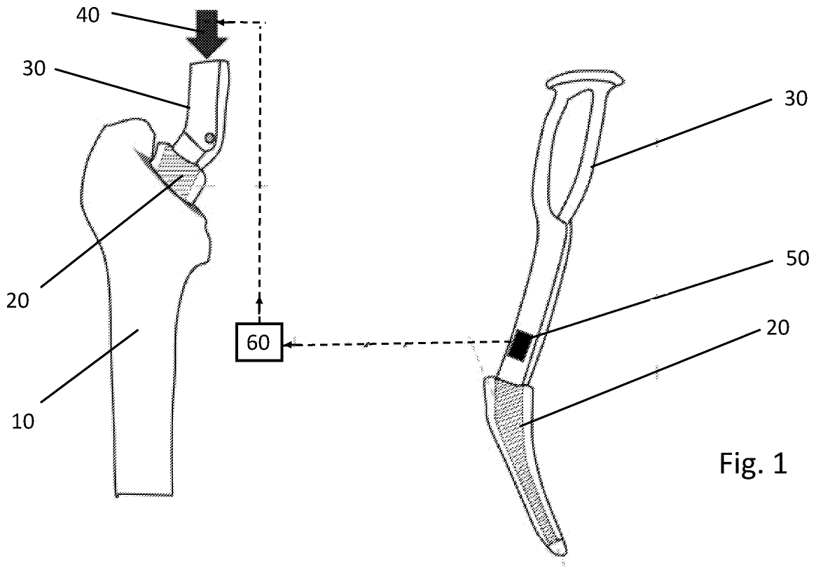

FIG. 1 shows an apparatus for inserting broaches and femoral stems in a bone (e.g. femur 10), in accordance with one example of the presently disclosed subject matter. The apparatus includes a broach 20 (or femoral stem, in the final insertion— herein after in the specification and claims: “broach”); a broach/stem handle 30; a real time, closed loop impactor 40 (schematically represented by an arrow); a sensor 50; and a processor 60, which are all operably connected to each other. Broach 20 may be a broach as known in the art, to prepare the femoral canal; and so may be broach handle 30. Processor 60 is configured to receive data from one or more sensors 50, analyze that data, whether explicitly or via deep learning, and provide instructions to impactor 40.

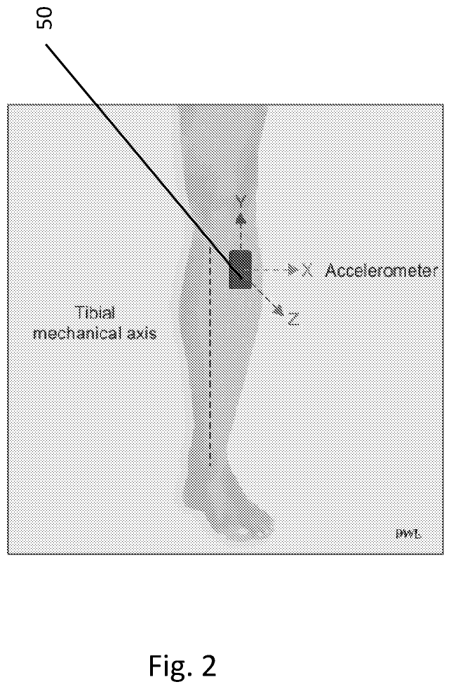

During a hip replacement procedure, in particular the process of inserting subsequently larger broaches into the femur, each broach 20 is impacted (hit) by impactor 40. The impact, such as resulting acoustic vibrations thereof, is sensed by sensor(s) 50. It is also possible to sense the change in the natural frequency of broach 20 and its damping coefficient, both indicative to the level of fixation of the broach/stem. The aforementioned features of the impact (or the acoustical or vibrational response of the bone due to the impact) are sensed by sensor(s) 50 and conveyed to processor 60, which includes an algorithm for analyzing those particular features with respect to bone stress. Processor 60 provides results of the algorithm's analysis in order to control the impactor, such as the rate and/or intensity of the impacts. In some examples, sensor(s) 50 is/are located between broach handle 30 and broach 20, as illustrated in FIG. 1; and in other examples, adjacent the knee, as illustrated in FIG. 2— or both. In other words, there may be more than one sensor 50 in more than one location, as well as more than one type, as understood from the description below. It is noted that the broach/stem area and knee area is expected to provide good acoustic coupling to the impacts.

Sensor 50 may include at least one of the following: an accelerometer, configured to measure a shock and after shock acceleration; a vibrometer, configured to measure a shock and after shock velocity; an interferometer, configured to measure a shock and after shock displacement (amplitude). The vibration has an amplitude (displacement) A; a velocity A*w, where w is 2*Pi*F of the vibration frequency F; and an acceleration A*w\hat{ }2. From this one can understand that for a given A, the position sensor is good at low frequency, the velocity sensor yields a better signal and intermediate frequency and the acceleration will give the strongest signal at high frequency.

Another possible sensor 50 is an acoustic emission sensor, configured to sense acoustic signals related to the generation of micro-cracks in the bone. The signal sensed by sensor 50 can be filtered to differentiate, using timing and/or frequency domain separation, between the impact signal and the signal from the tissue and bone, as known per se. In examples where sensor 50 is an acoustic emission device, which is sensitive to cracking in brittle materials, frequencies are typically in the tens and hundreds of kHz, sensitive to the generation of micro-cracks in the bone.

Yet another optional sensor 50 is one or more of a wide bandwidth microphone (including and exceeding the audible range), configured to measure an acoustic noise and spectrum generated by the impact; and transducers, configured to generate and detect acoustic waves, in which the transducer is excited at a desired frequency or frequency range and is coupled to broach 20. The vibrations propagated in broach 20, and waves reflected from the bone, are detected by the transducer. These reflected waves provide information about the bone stress.

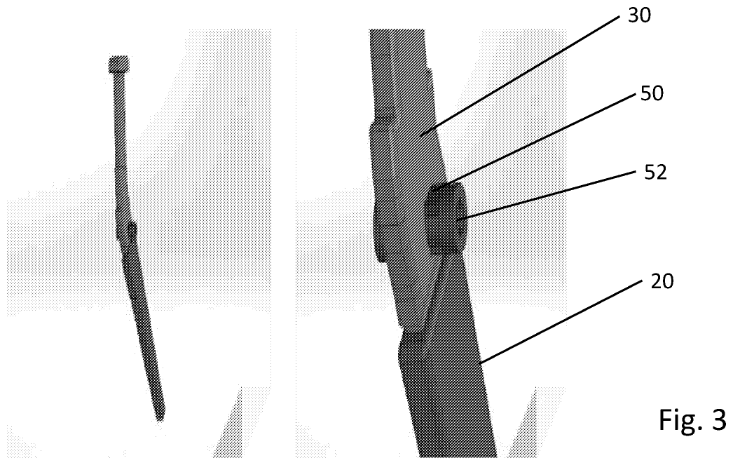

FIG. 3 illustrates sensor 50 held next to broach handle 20, by a sensor holder 52. Here, sensor 50 is not between broach handle 20 and broach 30, rather along the side of the broach handle. Other locations on broach handle 20 are also possible.

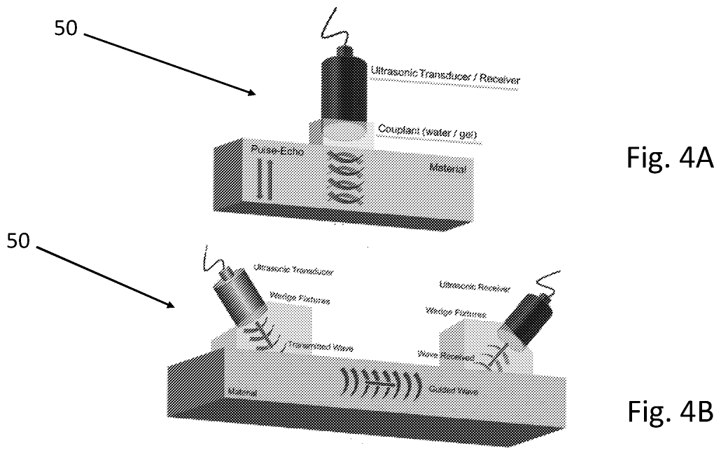

FIGS. 4A-4B show sensor 50 in the form of a transducer/receiver, in which the sensing can be in a transmitted mode and detection can be in a knee transducer, or via reflection where the detection is located on broach 20.

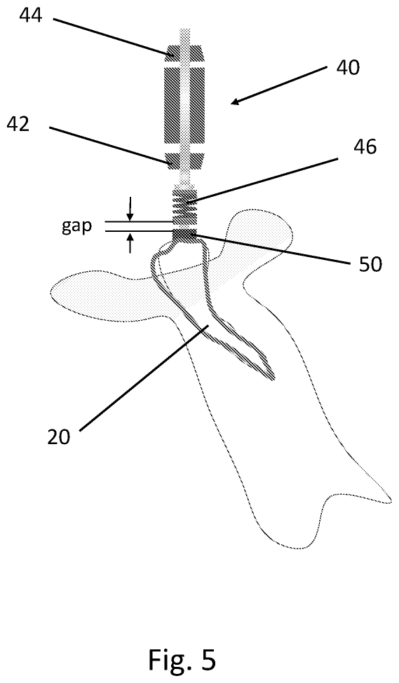

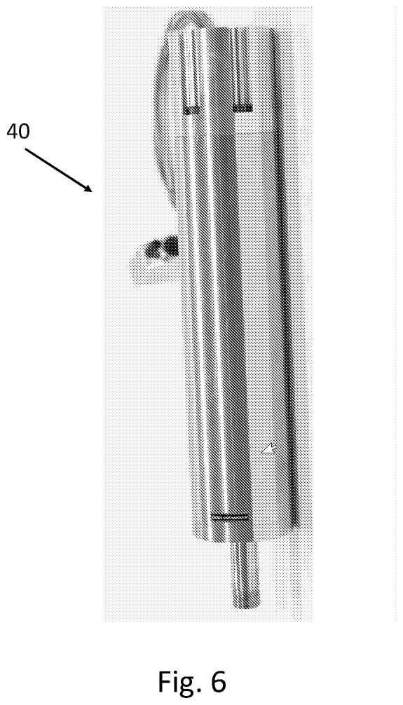

FIGS. 5-6 respectively show examples of impactor 40 in the form of an electro-magnetic impactor; and a piezo impactor. FIG. 5 illustrates impactor 40 constituted by an inertial impactor having two masses, namely, a lower/impacting mass 42 and an upper mass 44, designed to move in opposite directions where only one of the masses (the lower mass) operably hits broach 20. During impacting, lower mass 42 impacts a sandwiched spring 46, providing a force to sensor 50 and in turn, to broach 20. The stiffness of spring 46 determines the interaction time and the impact. Upper mass 44 and lower mass 42 may be actuated/moved in resonance, by a motor, a solenoid or a “Voice Coil” actuator, accelerating the lower mass to impact, via spring 46, which, due to its elasticity, preserves the energy transferred. Operating in resonance reduces the excitation energy required to the level required to compensate for the losses of the spring/lower mass/broach impact. The same action can be repeated with upper mass 44, impacting broach 20 in an opposite direction, applying force to extract the broach outwards. Regardless, in some examples, impactor 40 may include only one mass or be otherwise configured.

In some examples, masses 42 and 44 move synchronously in opposite directions providing an impactor design where the center of gravity (COG) is stationary to assure minimal fatigue and ease of use for the surgeon.

Lower mass 42 and upper mass 44 are made of a hard and rigid material so that the impact time is short. If the impact time, for example, is 10 micro-seconds (a typical interaction time of two solid bodies hitting each other), the mass is 200 grams, and the impact velocity is 1 m/sec, the resulting impact force equals 20 kN (according to F*t=M*V), which is within the range required in the art for broach/stem insertion.

The work performed is the force times displacement. As an example, 10 KN times 100 micron equals 1 Joule of work. With an impact rate of 10 impacts per see, the resultant mechanical power is 10 W. It is noted that a minimal travel (insertion) is required to overcome the elastic response of the bone tissue. Another consideration is that when the elastic zone of the bone is exceeded, a higher impact frequency can do the same work (travel) with a lower impact, reducing the vulnerability to bone fracture.

FIG. 6 illustrates impactor 40 constituted by a piezo-electric impact generator. As noted, impactor 40 may include a piezo-electric pulser (FIG. 6), for example, having accelerations of up to 500,000 m/s²; reproducible shocks (each impact is identical) of up to a 100 μm displacement; pulse widths of a few tenths of u-seconds; μs-exact triggering of the shock (impact); and repetition rates up to 100 Hz. Such a design also mitigates the impact that the surgeon feels.

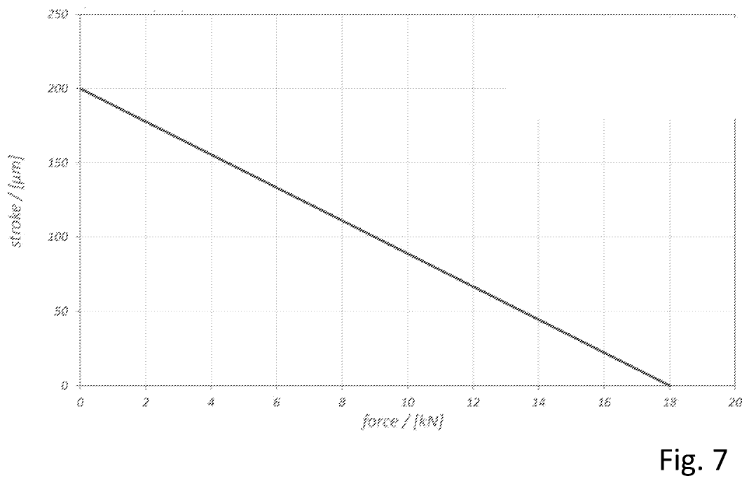

FIG. 7 depicts the relation between voltage, stroke, and impact force of the impactor of FIG. 6. The relationship between stroke and force is according to the line on the graph at various driving voltages operating the impactor. A driving voltage pulse of 250V will yield an impact stroke of 50 μm with a maximum pushing force of 13.5 kN. A voltage pulse of 750V will yield an impact stroke of 150 μm at a maximum pushing force of 4.5 kN. By setting the impactor voltage, one can provide the stroke amplitude and the force limit.

The presently disclosed subject matter is superior to the standard mallet as it optimizes and controls the impacting amplitude, force and frequency of the impacts, based on feedback from the bone as sensed by sensor(s) 50. Rather than manually impacting every 0.5 second or so, the apparatus and method can provide a 10 to 100 fold rate of impacting, thus minimizing the required impact level (force) and corresponding resultant bone stresses. In some examples, the subsequent impact is within the transient time of the previous impact, hence the friction is lower, with a dynamic rather than static coefficient of friction, as the broach continues to move or vibrate due to the previous impact during the consecutive impacts in a high frequency series of impacts.

During insertion, broach or stem 20 and bone 10 are in contact, whereby the bone interacts with the broach and affects the returning waves, which has been realized during trials, and a wave propagation simulation, to infer the bone stress.

In some examples, an AI system is incorporated in processor 60 to determine when critical bone stress is being approached by the impacting.

Sensor(s) 50 sense(s) each impact and provide raw data for the early detection of approaching the optimal insertion, and control respectively the impact frequency and intensity (force).

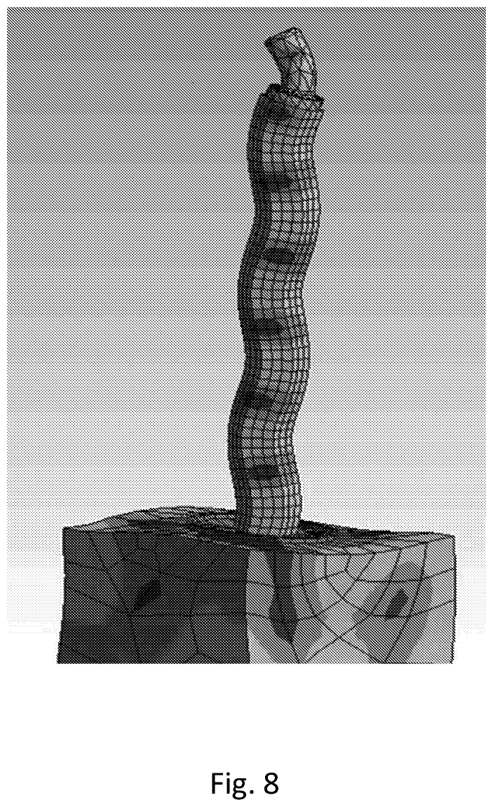

FIG. 8 shows a snap-shot of a simulation of an impact on a broach/stem, showing vibration waves in the broach/stem and the bone. The darker areas represent compaction and the lighter areas represent expansion of the broach and the bone, showing the bending of the broach/stem.

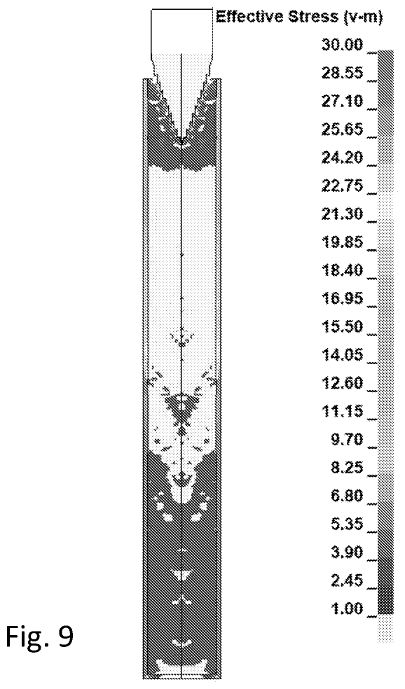

FIG. 9 shows a snap-shot of a simulation video showing bone reaction to the broach upon an impact, Von Mises stress is shown. The stress in the bone is simulated and is compared to known bone properties, to estimate if the bone is close to a critical stress. Stresses below 1 MPa are not shown in order to show the traveling wave more clearly. The stress wave moves mainly in the cortical bone, while the trabecular (soft inner tissue) stresses are more localized, since it is a softer material than the cortical bone. Shock waves propagating in the bone illustrate the stress and compression upon impact. Shock waves can be assessed by measuring the reflection of the transducer (sensor 50). The measurement is challenging as the density of broach 20 is much higher than that of the bone and the bone does not greatly affect the broach. It is possible to detect and measure the shock waves by smart signal processing. In some examples, artificial intelligence (AI) is used to teach the system algorithm.

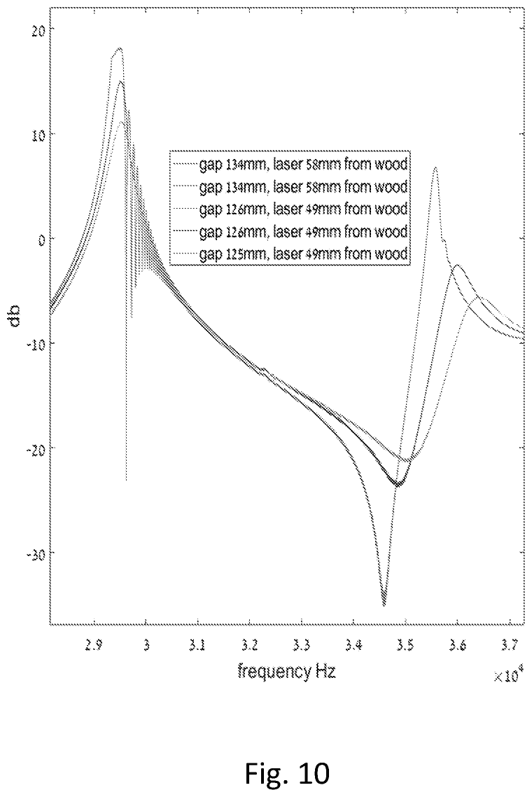

FIG. 10 shows a graph of wave vibration measured in decibels as a function of frequency— the bone is simulated by wood. In this example, a transducer sensor is sensing the change in conditions via the change in measured natural frequency and damping. The term “gap” refers to the depth that the broach/stem is inserted. The deeper the insertion, the more firmly the broach/stem is held in the bone and the natural broach frequency and the damping increases. Upon emergence of cracks, the bone stem is held less firmly. Thus, the measurement of wave vibration provides a way to detect the threshold limit of force prior to the bone cracking, using the known natural frequency of each broach.

The invention method involves monitoring the excitation in a longitudinal vibration and/or bending vibration to sense the bone stress. The aforementioned method is shown with the transducer signal, but the same mechanism applies with other sensors. The simulation helps predict the bone stress, via reflected signals.

It should be understood that the above description is merely exemplary and various examples of the present presently disclosed subject matter may be devised, mutatis mutandis, and that the features described in the above-described examples, and those not described herein, may be used separately or in any suitable combination; and the presently disclosed subject matter can be devised in accordance with examples not necessarily described above.

1. An apparatus for inserting a broach or stem into a bone, the broach and stem having an associated handle for impaction, the apparatus comprising:
a closed loop impactor configured to provide a series of impacts on the handle;
at least one sensor configured to sense the series of impacts, including the force and vibration frequency of the impacts and to provide real-time feedback of a condition of the bone; and
a processor operably connected to the at least one sensor and configured to receive the real-time feedback therefrom, and operably connected to the impactor to control the rate and force of the impacts that the impactor applies to the broach.

2. The apparatus of claim 1, wherein the sensor is any one or combination of an accelerometer, a vibrometer, an interferometer or position sensor, a wide band microphone, an acoustic emission sensor, and a transducer.

3. The apparatus of claim 1, wherein the sensor is configured to provide real-time feedback based on shock-induced vibrational data or acoustic data.

4. The apparatus of claim 1, wherein the processor includes an artificial intelligence system for multi-parameter data analysis.

5. The apparatus of claim 1, wherein the impactor is a piezoelectric impactor.

6. The apparatus of claim 1, wherein the impactor is an electro-magnetic impactor.

7. The apparatus of claim 1, wherein the impactor is configured to provide impacts at a frequency of between 10 and 100 impacts per second.

8. The apparatus of claim 1, wherein the impactor comprises a moving mass, a spring, a hitting mass and an excitation device.

9. The apparatus of claim 7, wherein the excitation device is either a motor; a solenoid; or a voice coil.

10. The apparatus of claim 1, wherein the at least one sensor is disposed between the broach handle and the broach.

11. The apparatus of claim 1, wherein the at least one sensor is disposed on one or more sides of the broach handle and/or the broach.

12. The apparatus of claim 1, wherein the at least one sensor is disposed on or adjacent a corresponding knee.

13. The apparatus of claim 1, wherein the impactor is configured to impact the broach or bone at a rate of 1 Hz to 100 Hz.

14. A method of inserting a broach or stem into a bone using an impactor, the method comprising:
impacting the broach or stem into the bone;
using at least one sensor to sense signals (a) produced by the broach or stem during said impacting, or (b) signals generated in the bone as a result of the impacting;
analyzing said signals via an algorithm operated by a processor; and
providing real-time feedback to control the force and rate of impacting.

15. The method of claim 14, wherein the sensor is an acoustic emission sensor, configured to sense acoustic signals related to the generation of micro-cracks in the bone, and signal sensed by the acoustic emission sensor is filtered to differentiate between the impact signal and the signal from the tissue and bone.

16. The method of claim 14, wherein the impacting is performed so that subsequent impacts are within the transient time of a previous impact, thereby resulting in a dynamic coefficient of friction to lower friction between the broach or stem and the bone.

17. The method of claim 14, wherein the impacting comprises using an electro-magnetic impactor and/or a piezo impactor.

18. The method of claim 14, wherein the impacting comprises using an inertial impactor having a lower/impacting mass and an upper mass designed to move in opposite directions where only the lower mass operably impacts the broach or stem.

19. The method of claim 14, wherein the impacting is performed at a rate of 1 Hz to 100 Hz.
